(19) United States
(12) Patent Application Publication
(10) Pub. No.: US 2003/0167967 A1
(43) Pub. Date: Sep. 11, 2003

(54) Glass ionomers for enhancing mineralization of hard tissue

(76) Inventors: Timo Narhi, Helsinki (FI); Antti Yli-Urpo, Littoinen (FI); Helena Yli-Urpo, Piikkio (FI); Joni Korventausta, Turku (FI); Mika Jokinen, Turku (FI)

Correspondence Address: James C. Lydon, Suite 100, 100 Daingerfield Road, Alexandria, VA, 22314, US

(21) Appl. No.: 10/084,999

(22) Filed: Mar. 1, 2002

(51) Int. Cl. A61K 6/06; A61K 6/08

(52) U.S. Cl. 106/35; 623/23.62; 523/113; 523/114; 523/115; 523/116; 501/1; 501/16; 501/17

(57) ABSTRACT

The invention relates to glass ionomer for enhancing mineralization of hard tissue of mammals. The glass ionomer comprises, a inert biocompatible ceramic and a bioactive ceramic. The invention also relates to a method for enhancing mineralization of hard tissue of mammals with the aid of said glass ionomer. The invention further relates to the use of said glass ionomer for the preparation of products intended for treatment of defects of soft and hard tissue as well as the use of said glass ionomer for the preparation of a variety of products wherein the ability to enhance mineralization is advantageous.

### FIELD OF INVENTION

The present invention relates to novel bioactive glass ionomers that release Si, Ca, and P ions and induce CaP (i.e. compounds of calcium and phosphate) deposition on mineralized tissues in a controllable manner.

### BACKGROUND OF THE INVENTION

The publications and other materials used herein to illuminate the background of the invention, and in particular, cases to provide additional details respecting the practice, are incorporated by reference.

Glass ionomers have been used as filler material in various tooth restorations. Glass ionomers contain fluoroaluminosilicate glass, and they are set with polymer acid, e.g polycarboxylic acid. In an acidic environment glass granules release Ca²⁺ ions, which bond with O⁻ groups. After the setting reaction has completed the material is bard and insoluble in the human body. Fluoride gets slowly released from the bulk material and strengthens the surrounding apatite. Glass ionomers bond directly with the apatite and no separate bonding agents are needed. Glass ionomers have also been used for the fixation of orthopedic devices.

However, glass ionomers cannot increase tooth or bone mineralization. Controlled release of ions needed for mammal hard tissue mineralization is an essential prerequisite for a bioactive glass ionomer.

A tailor-made controlled release of ions is needed to orientate the growth of hard and soft tissue.

### OBJECT AND SUMMARY OF THE INVENTION

One object of the present invention is to provide glass ionomers for enhancing mineralization of hard tissue of mammals. Another object is to provide a method using said glass ionomer for enhancing mineralization of hard tissue of mammals. Yet another object is to provide said glass ionomer, which would rapidly and safely release ions needed for calcium phosphate formation in the tissue environment in contact with said glass ionomer, for use in repairing hard and/or soft tissue defects in mammals.

Thus this invention provides glass ionomers for enhancing mineralization of hard tissue of mammals comprising an inert biocompatible ceramic and a bioactive ceramic.

This invention further provides a method for enhancing mineralization of hard tissue of mammals using said cement. The steps of the method comprise

a) mixing a bioactive ceramic with an inert biocompatible ceramic to obtain a glass ionomer material,

b) adding a polymer acid and/or polyacid modified resin to said material obtained in step a) to initiate a setting reaction by dissolving and nucleating Ca²⁺ ions and/or by a polymerization reaction

c) applying said mixture obtained in step b) to where said glass ionomer is to be used for enhancement of mineralization of hard tissue of mammals, and

d) letting said mixture set,

wherein said set glass ionomer enhances mineralization of hard tissue in contact with it.

Yet another aspect of this invention concerns the use of said glass ionomer for the preparation of products intended for treatment of defects of hard and/or soft tissue, preferably maxilla, mandible, tooth, root canal, pulp of tooth, gingival, ear, nose, skull, joints, defects in bone and/or subcutaneous soft tissue, most preferably for periodontal use. A further aspect of this invention concerns the use of a said glass ionomer for the preparation of products selected from the group consisting of implant materials, tissue coating materials, reconstructive parts for tissues, bone augmentation materials and scaffolds for tissue engineering. Yet further aspects of this invention concern the use of a said glass ionomer for the production of injectable material, preferably a solution or suspension; material used for coating of teeth and bone; and dental products used as root canal filling of tooth or a cavity of a tooth or root of a tooth, as tooth pulp capping material, as cementing material of temporary crowns, or for periodontal defects.

### BRIEF DESCRIPTION OF THE FIGURES

FIG. 1 shows release of Si ions from four different bioactive glass containing glass ionomers and two different conventional glass ionomers as a function of time when immersed in simulated body fluid at 37° C. for 1, 6, 24, 72, 168 and 336 hours, wherein:

A=autopolymerizing glass ionomer with 0 wt-% of bioactive glass;

B=autopolymerizing glass ionomer with 10 wt-% of bioactive glass;

C=autopolymerizing glass ionomer with 30 wt-% of bioactive glass;

D=light curing glass ionomer with 0 wt-% of bioactive glass;

E=light curing glass ionomer with 10 wt-% of bioactive glass and

F=light curing glass ionomer with 30 wt-% of bioactive glass.

FIG. 2 shows precipitation of P ions released from four different bioactive glass containing glass ionomers and two conventional glass ionomers as a function of time when immersed in simulated body fluid at 37° C. for 1, 6, 24, 72, 168 and 336 hours.

FIG. 3 shows precipitation of Ca ions released from four different bioactive glass containing glass ionomers and two conventional glass ionomers as a function of time when immersed in simulated body fluid at 37° C. for 1, 6, 24, 72, 168 and 336 hours.

FIG. 4 shows release of Si ions from two different bioactive glass ionomers comprising Ca and P containing silica gel (Si-gel) and a conventional glass-ionomer cement as a function of time when immersed in simulated body fluid at 37° C. for 0, 6, 27, 48, 73, 124, 171, 248 and 336 hours wherein:

A=autopolymerizing glass-ionomer with 30 wt-% of Ca and P containing Si-gel;

B=autopolymerizing glass-ionomer with 10 wt-% of Ca and P containing Si-gel;

C=autopolymerizing glass-ionomer with 0 wt-% of Ca and P containing Si-gel.

FIG. 5 shows precipitation of P ions released from two different bioactive glass ionomers comprising Ca and P containing silica gel and a conventional glass ionomer as a function of time when immersed in simulated body fluid at 37° C. for 0, 6, 27, 48, 73, 124, 171, 248 and 336 hours.

FIG. 6 shows precipitation of Ca ions released from two different bioactive glass ionomers comprising Ca and P containing silica gel and a conventional glass ionomer as a function of time when immersed in simulated body fluid at 37° C. for 0, 6, 27, 48, 73, 124, 171, 248 and 336 hours.

FIG. 7 shows the growth of yeast cells in contact with bioactive glass containing glass ionomer and conventional glass ionomer defined as above for FIG. 1.

FIG. 8 shows a scanning electron micrograph (SEM) of CaP depositions on bioactive glass containing glass ionomer with 30 wt-% of bioactive glass (S53P4) after 336 hours of immersion in simulated body fluid.

FIG. 9 shows a SEM picture of CaP depositions on resin reinforced bioactive glass containing glass ionomer with 30 wt-% of bioactive glass (S53P4) after 336 hours of immersion in simulated body fluid.

FIG. 10 shows an electron-dispersive X-ray analysis (EDXA) from the surface of bioactive glass containing glass ionomer with 30 wt-% of bioactive glass (S53P4) after 336 hours of immersion in simulated body fluid showing Si, Ca, P peaks.

FIG. 11 shows an EDXA picture from the surface of a resin reinforced bioactive glass containing glass ionomer cement with 30 wt-% of bioactive glass (S53P4) after 336 hours of immersion in simulated body fluid showing Si, Ca, P peaks.

FIG. 12 shows a SEM picture of mineralized canine dentin tubules under a tooth cavity filled with bioactive glass containing glass ionomer after 6 weeks.

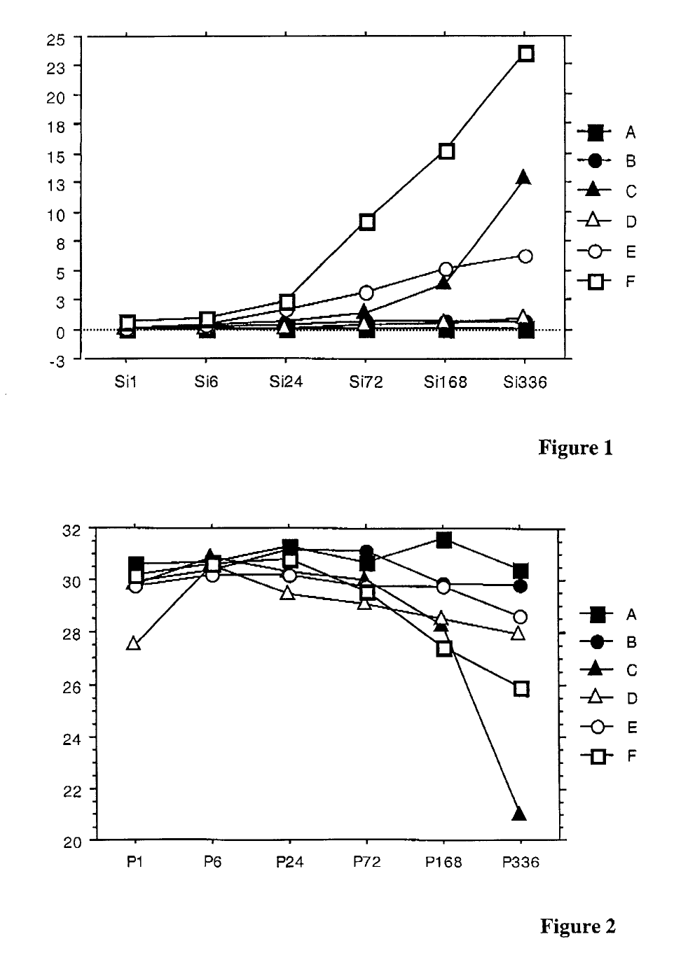

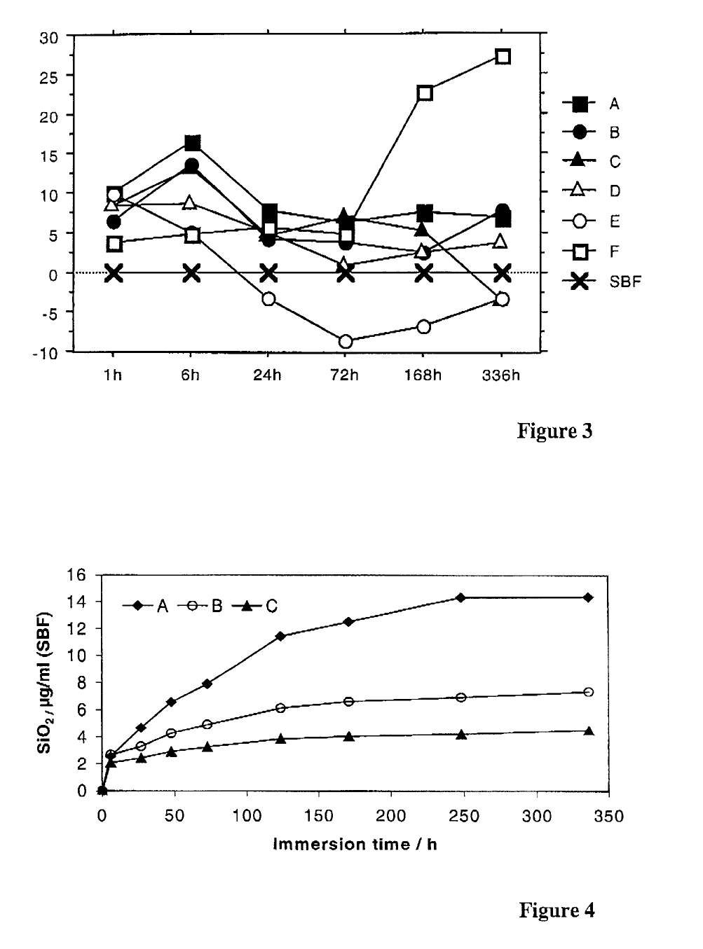

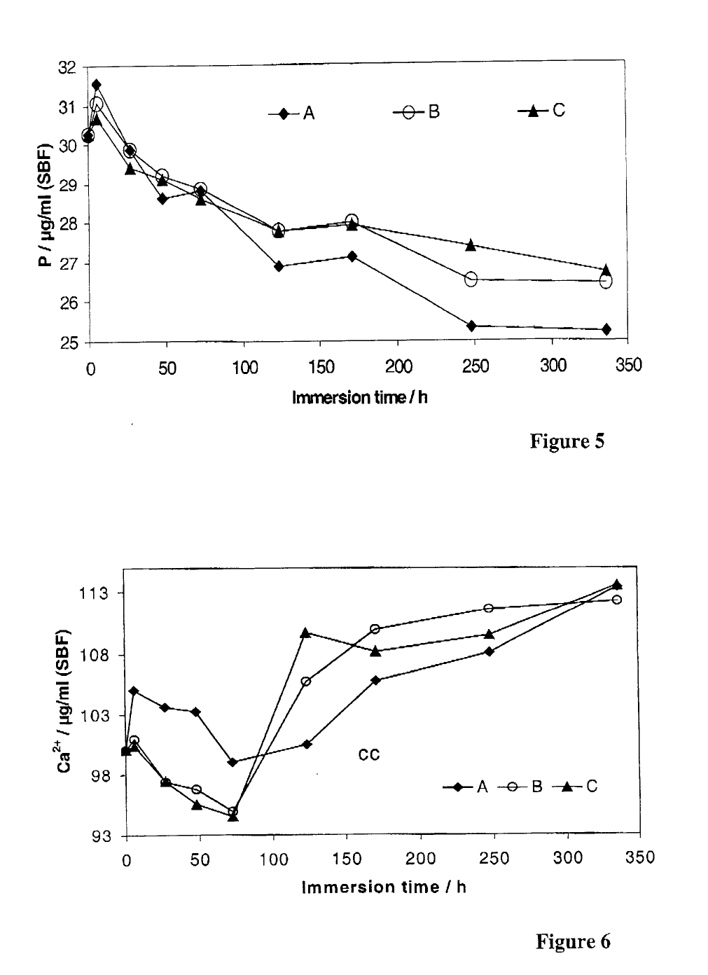

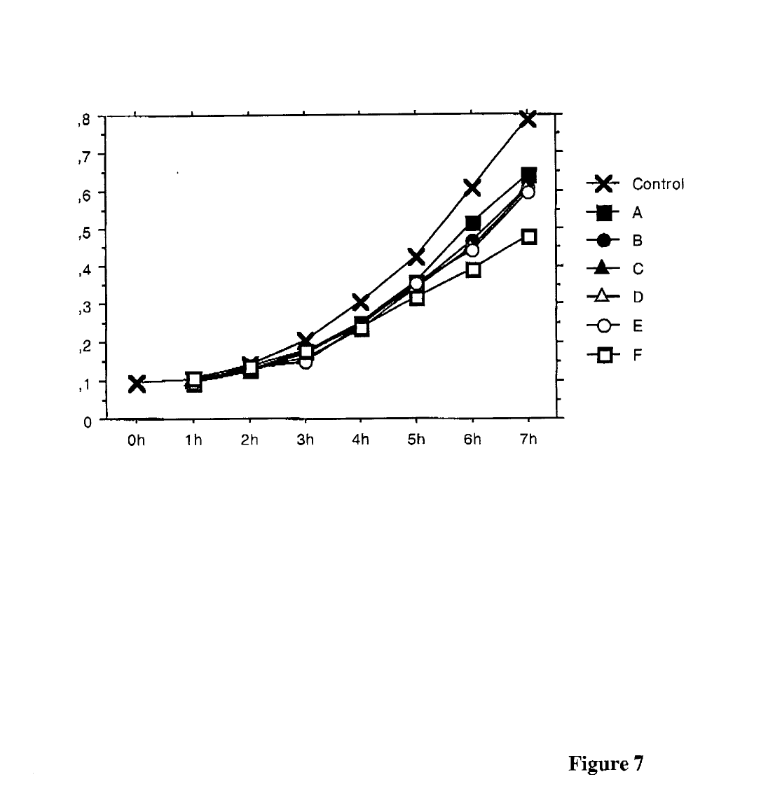

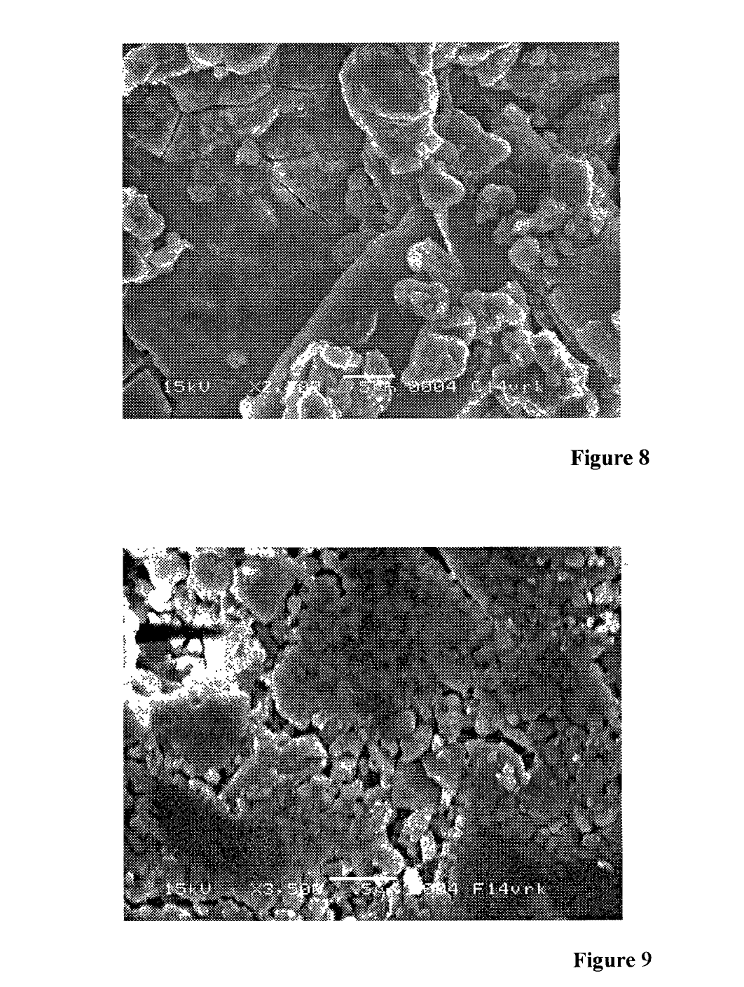

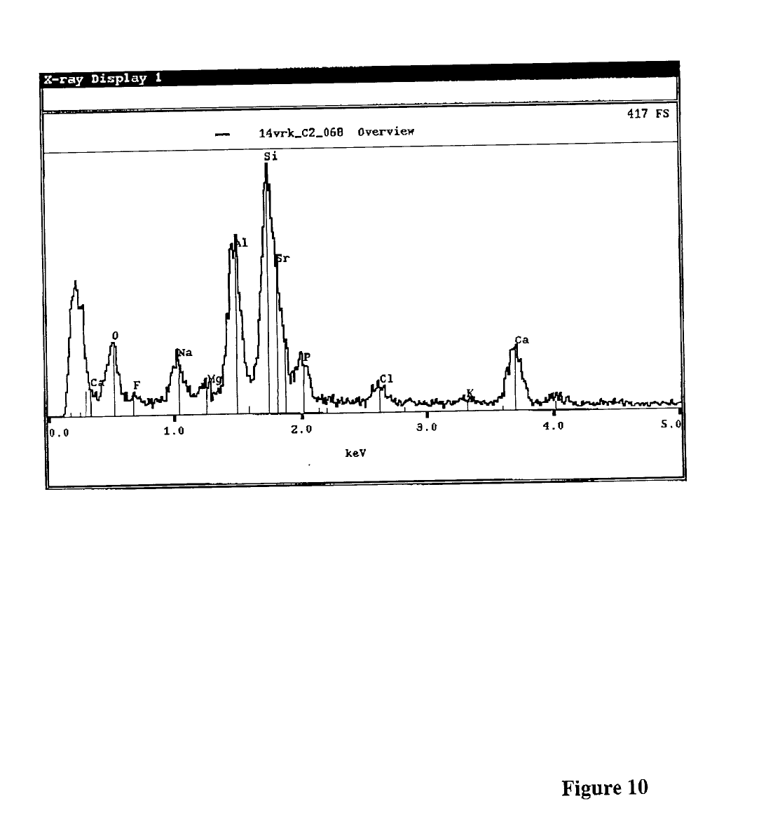

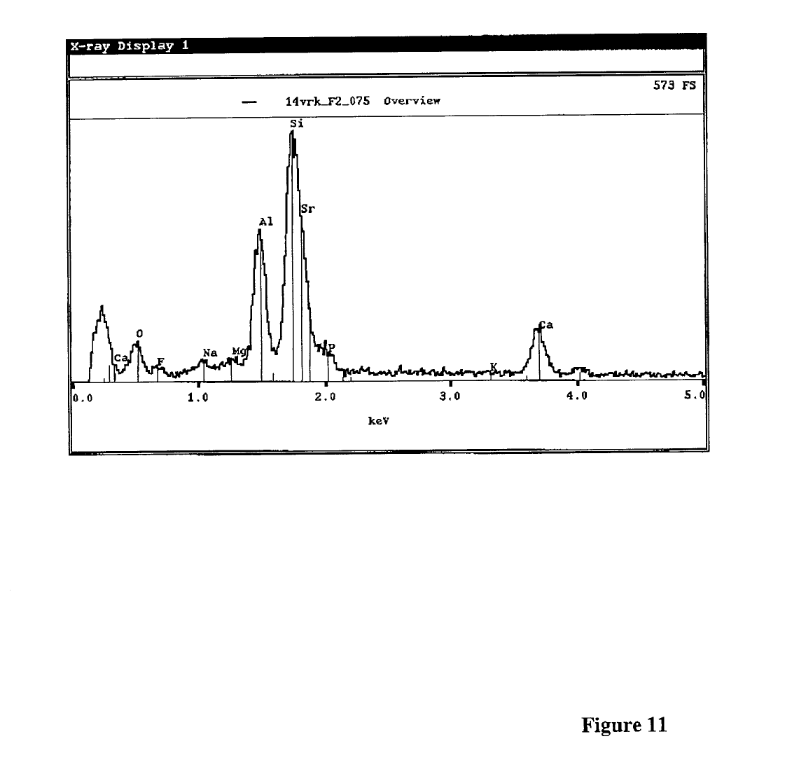

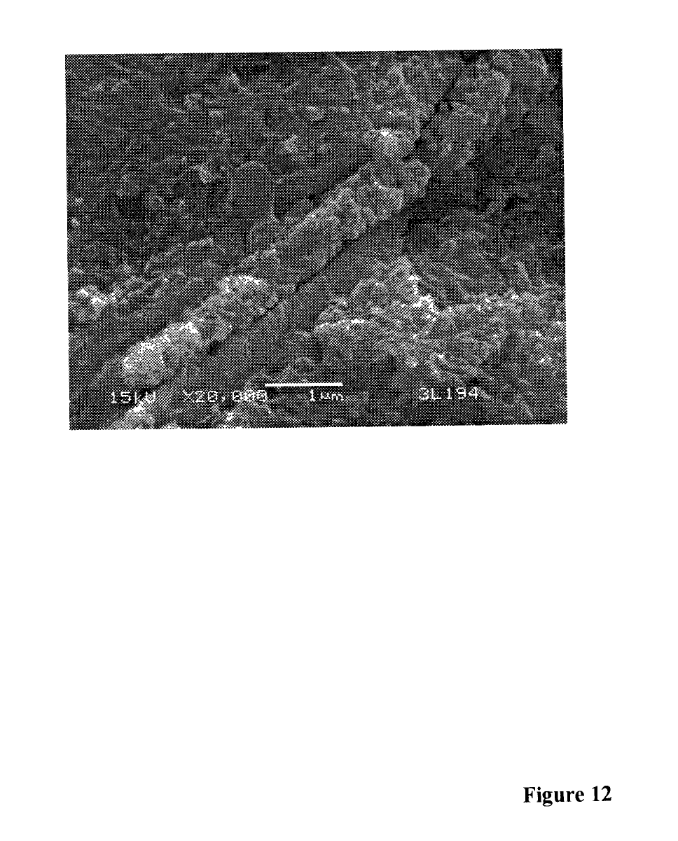

### DETAILED DESCRIPTION OF THE INVENTION

The term “inert” refers in the context of this application to component or particle that does not in an aqueous environment release in an essential amount active agents from the cement.

The term “biocompatible” in the context of this application means that the component or particle is compatible with the other ingredients of the glass ionomer and is not deleterious to the recipient thereof,

The term “bioactive ceramic” in the context of this application refers to a material that elicits a specific biological response at the interface of the material by enhancing mineralization of the tissue in contact with the bioactive ceramic comprised in the glass ionomer of the invention.

The term “bioactive agent” refers in the context of this application to a material that can elicit a local and/or systemic specific biological response in the tissue and/or organism which it is brought in contact with, which response significantly differs from any response possibly obtained without incorporation of said bioactive agent.

The term “inert biocompatible ceramic” in the context of this application refers to biocompatible ceramic that does not elicit a specific biological response at the interface of the material, which response would comprise significant enhancement of mineralization of the tissue in contact with it.

Glass ionomers are materials, which comprise an acid-soluble fluoroaluminosilicate glass. These set by an acid-base reaction using an aqueous polyacid liquid in the presence of water. In the context of this application glass ionomers also refer to resin-modified glass ionomers, polyacid-modified resin composites (compomers), ionomer-resin suspensions and composite resins that comprise fluoroaluminosilicate glass.

Resin-modified glass ionomers (RMGI) are also referred to as reinforced glass ionomers (RGI's) or resin-ionomers. These are glass ionomer materials, which consist of a matrix of acidic and polymerizable polymers, which set by both acid/base and polymerization reactions.

Polyacid-modified resin composites (PMRC) consist of glass ionomer components and a polymerizable resin matrix. They may or may not be hydrophilic. These materials are anhydrous and set by a polymerization reaction.

Ionomer-resin suspensions (IRS) are also referred to as fluoride releasing resins (FRR). These usually contain a fluoroaluminosilicate glass suspended in a resin matrix, which sets by a polymerization reaction.

Composite resins are also referred to as composites or filled resins. Composite resins consist of inert glass or quartz filler in a resin matrix. These set by a polymerization reaction.

The term “sol-gel derived ceramic material” refers in the context of this application to any ceramic material obtainable by a sol-gel process that can release ions needed for apatite formation.

The term “tissue defect” refers to any site or locus being deficient in hard tissue components anatomically normal to the site of the body of said mammal often also surrounded by different soft tissues and/or body fluids.

This invention concerns glass ionomer for repairing hard and/or soft tissue defects in mammals. Characteristic for the glass ionomer is that it comprises water-reactive bioactive ceramic particles (e.g. bioactive glass or sol-gel-derived ceramic material), and non-reactive filler particles, which can be used to tailor the mechanical properties of the material. Typically a polymer acid, (e.g. polycarboxyl acid, acrylic acid, maleic acid, tartaric acid or their copolymer or any combination thereof), is added prior to use to control the setting reaction of the said glass ionomer.

The present invention provides a biologically acceptable material, i.e. a glass ionomer, that can be injected or implanted into a mammal including humans said material comprising a mixture of bio-compatible bioactive glass ceramic powder and an inert glass ceramic powder, which typically can be made to set after mixing with a polymer acid, e.g. polycarboxyl acid. Typically said glass ceramic powder is a mixture of bioactive glass powder or Ca and P doped sol-gel-derived silica particles and fluoroaluminosilicate glass powder.

The inert biocompatible ceramic can preferably be calcium fluoroaluminosilicate glass optionally comprising oxides of alkali metals, alkali-earth metals, boron, phosphorous titanium, polymerizable matrix material, photoinitiator and/or reducing agent or any combination thereof.

The bioactive ceramic can preferably be bioactive glass and/or a sol-gel derived ceramic material.

If the bioactive ceramic is a bioactive glass it can preferably comprise oxides of silicon, alkalis, alkaline earths and optionally other elements such as aluminum, boron and phosphorous wherein said oxides are present in the following amounts:

| | |
|---|---|
| SiO₂ | 38-57.5 wt %, |
| Na₂O | 16-29 wt %, |
| CaO | 11-25 wt %, |
| Al₂O₃ | 0-3 wt %, |
| B₂O₃ | 0-3 wt %, and |
| P₂O₅ | 0-8 wt %. |

Most preferred bioactive glasses are glasses S38P8, S45P7, S46P0, S48P2, S51P7, S52P8, S53P4, S55.5P4, S56P6 and S57.5P5 specified in more detail in example 8.

The bioactive ceramic is preferably a sol-gel derived silica gel and it can optionally comprising any one or several of elements consisting of Al, B, Ca, F, P, K, Mg, N and Ti. The bioactive ceramic preferably comprises oxides of silicon, alkalis, alkaline earths and other elements such as phosphorous wherein said components are present in the bioactive ceramic in the following amounts:

| | |
|---|---|
| SiO₂ or Si-gel | 1-100 wt %, |
| Na₂O | 0-45 wt %, |
| K₂O | 0-45 wt % |
| CaO | 0-40 wt %, |
| MgO | 0-40 wt %, and |
| P₂O₅ | 0-60 wt %. |

The particle size of the powder of the cement is 0.01-6 000 μm, preferably 0.1-400 μm, most preferably <45 μm. The bioactive ceramic preferably is a powder with a particle size of <400 μm most preferably including particles in the size range of 1 to 45 μm.

The powder of the glass ionomer can optionally contain one or more active, i.e. physically, chemically and/or bioactive, or inactive agents such as drugs or antimicrobial agents, growth factors, preservatives, coloring, flow enhancing, reinforcing, bonding or suspension enhancing agents. Active agents can be added in various forms e.g. granules, fibers, nets or microspheres. The ratio of the glass powder and polymer acid that can be added to initiate setting is such that the material remains homogenous during the application procedure and sets in the target tissue.

Bioactive glass or sol-gel derived silica particles retain their bioactive properties within the material after the setting reaction has completed. Bioactive particles begin to dissolve as the water content of the glass ionomer increases, which leads to dissolution of ions needed for mineralization of bone, cartilage, dentin or enamel or the glass ionomer itself.

The glass ionomer can be used in reconstruction or augmentation of mammal hard tissue structures in a patient in need thereof comprising inserting, e.g. by injecting or packing the material into tissue defects.

Anatomic structures treatable according to the method of this invention include, but are not limited to, maxilla, mandible, tooth, root canal, and defects in bone and joints, periodontal lesions or for plastic surgery purposes.

The polymer acid, which can be mixed with the glass ionomer to bring about the mixture to be applied for use, can be polycarboxyl acid, acrylic acid, maleic acid or tartaric acid or their copolymer or any combination thereof.

The glass ionomer according to the invention can also comprise bioactive agents other than bioactive glass, e.g. anti-inflammatory agents, antimicrobial agents, corticosteroids, fluoride, growth factors, heparin, hydroxylapatites, ormosiles, silica gel, tooth whitening agents, vitamins, and/or living cells. The bioactive agent can be mixed with an inert non-soluble agent.

The glass ionomer according to the invention can be used for the preparation of products intended for treatment of defects of soft and hard tissue, e.g. maxilla, mandible, tooth, root canal, ear, nose, skull, joints, defects in bone. The glass ionomer can be a dental product used as root canal filling of a tooth or a cavity of a tooth, as cementing material of temporary crowns, of orthopedic and dental implants.

To obtain a glass ionomer according to the invention bioactive glass or alternatively Ca and P doped sol-gel derived silica granules or powder can be mixed with fluoroaluminosilicate glass powder to achieve a homogenous mix. The powder with bioactive particles and inert glass can then be mixed with polymer acid (e.g. polycarboxyl acid, acrylic acid, maleic acid, tartaric acid or their copolymer or any combination thereof), which initiates the setting reaction by dissolving and nucleating the Ca²⁺ ions. Alternatively or concurrently a polyacid modified resin can be used. Speed of the setting reaction as well as final hardness of the set material can be adjusted by changing the filler content and composition.

In the aqueous environment bioactive particles starts to dissolve releasing Si, Ca and P ions into the surrounding environment. The dissolved ions precipitate on the surrounding tissue surfaces forming CaP layers or plugs.

The following examples are offered as illustrations of the present invention and are not to be constructed as limitations thereof. Example 1 and 2 disclose examples on how to prepare a bioactive glass ionomer. Example 3 discloses the preparation and use of cement according to the invention. Examples 4a, 4b and 4c demonstrate how dissolving of the bioactive glass, which can be a component of the glass ionomer of the invention, releases Si, Ca and P ions in simulated body fluid. Examples 5 and 6 disclose glass ionomers using different bioactive agents as components of the cement.

### EXAMPLES

### Example 1

Bioactive glass ionomers were prepared by making a homogenous mixture of bioactive particles and inert ceramic powder.

TABLE 2
Possible, preferred and most preferred
compositions of bioactive glass ionomers

| | Bioactive component | Inert component |
|---|---|---|
| Possible | 1-99 w %; | 99-1 w %; |
| Preferred | 1-50 w % | 99-50 w %; |
| Most preferred | 1-30 w % | 99-70 w % |

The homogenous powder of bioactive and inert particles is mixed with a polymer acid (e.g. polycarboxyl acid or copolymer of acrylic acid and maleic acid), which dissolves Ca²⁺ ions from the powder. Ca²⁺ ions form compounds with the unoccupied OH⁻ groups, which leads to the setting of the glass ionomer in question. Speed of the setting reaction can be adjusted from few seconds up to several minutes by varying the specific composition of the glass ionomer.

### Example 2

Bioactive glass ionomer powder is mixed as described in the example 1, except that the inert powder is first mixed with a resin component. A resin can be added to improve mechanical properties of the material or to make the material light curing.

### Example 3

Bioactive glass ionomer powder is mixed as described in example 1, except that the inert ceramic component is first mixed with an active agent (e.g. growth factor, antibiotic) in order to make a material that can release admixed agents in a well-controlled manner.

### Example 4

Bioactive glass ionomer is first mixed as described in example 1. The glass ionomer powder is then mixed with a polymer acid to a paste after which the material is packed into a tooth cavity. The material sets in situ and releases Ca, P, and Si ions that initiate the formation of calcium phosphate crystals within dentin tubules. Depending on the composition of the bioactive component the material may have antimicrobial properties against microorganisms in close contact with the surface of the material.

### Example 5

Bioactive glass ionomer is mixed as described in example 4, except that the amount of acid and/or water is higher, which makes the material less viscous. Low viscosity material can be used as a liner in deep cavities under conventional filling materials or for temporary releasing during operations only to protect against irritations and/or for mineralizing tissues like tooth or bone or to control microbial contaminations in the operation areas, wounds, gingiva, skin, mucosa or bone.

### Example 6

Bioactive glass ionomer is mixed as described in example 4, except that the material is used as a temporary filling only to increase the mineral content of the dentin and enamel. Increased mineral content increases the bond strength between tooth and bonding agents. This significantly improves the bond between the tooth and ceramic or cement filling materials and crowns or fixed partial dentures.

### Example 7

Bioactive glass ionomer is mixed as described in the example 4. The glass ionomer is then used for cementing titanium and/or polymer implant devices (e.g. hip prostheses) into a body of a mammal.

### Example 8

Bioactive glasses suitable for the glass ionomer of the invention can for example have the following composition by weight percentage (wt-%):

| Glasstype | Na₂O | CaO | P₂O₅ | B₂O₃ | Al₂O₃ | SiO₂ |
|---|---|---|---|---|---|---|
| S57.5P5 | 16.00 | 18.00 | 5.00 | 3.00 | 0.50 | 57.50 |
| S56P6 | 19.00 | 16.00 | 6.00 | 1.50 | 1.50 | 56.00 |
| S51P7 | 20.00 | 17.00 | 7.00 | 3.00 | 2.00 | 51.00 |
| S53P4 | 23.00 | 20.00 | 4.00 | 0.00 | 0.00 | 53.00 |
| S45P7 | 24.00 | 22.00 | 7.00 | 2.00 | 0.00 | 45.00 |
| S52P8 | 25.00 | 12.00 | 8.00 | 0.50 | 2.50 | 52.00 |
| S46P0 | 26.00 | 25.00 | 0.00 | 2.00 | 1.00 | 46.00 |
| S38P8 | 27.00 | 23.00 | 8.00 | 1.00 | 3.00 | 38.00 |
| S48P2 | 28.00 | 19.00 | 2.00 | 1.50 | 1.50 | 48.00 |
| S55.5P4 | 29.00 | 11.00 | 4.00 | 0.00 | 0.50 | 55.50 |

It will be appreciated that the methods of the present invention can be incorporated in the form of a variety of embodiments, only a few of which are disclosed herein. It will be apparent for the specialist in the field that other embodiments exist and do not depart from the spirit of the invention. Thus, the described embodiments are illustrative and should not be construed as restrictive.

1. Glass ionomer for enhancing mineralization of hard tissue of mammals comprising an inert biocompatible ceramic and a bioactive ceramic.

2. The glass ionomer according to claim 1 wherein the glass ionomer comprises 1 to 99 wt-% of said inert biocompatible ceramic and 1 to 99 wt-% of said bioactive ceramic.

3. The glass ionomer according to claim 2 wherein the glass ionomer comprises 50 to 99 wt-% of said inert biocompatible ceramic and 1 to 50 wt-% of said bioactive ceramic.

4. The glass ionomer according to claim 3 wherein the glass ionomer comprises 70 to 99 wt-% of said inert biocompatible ceramic and 1 to 30 wt-% of said bioactive ceramic.

5. The glass ionomer according to claim 1 wherein said inert biocompatible ceramic is a glass ionomer, resin-modified glass ionomer, polyacid-modified resin composite, ionomer-resin suspension or a composite resin comprising fluoroaluminosilicate glass.

6. The glass ionomer according to claim 1 wherein said inert biocompatible ceramic is calcium fluoroaluminosilicate glass optionally comprising oxides of alkali metals, alkali-earth metals, boron, phosphorous titanium, polymerizable matrix material, photoinitiator and/or reducing agent or any combination thereof.

7. The glass ionomer according to claim 1 wherein said bioactive ceramic is bioactive glass and/or a sol-gel derived ceramic material.

8. The glass ionomer according to claim 1 wherein said bioactive ceramic is bioactive glass comprising oxides of silicon, alkalis, alkaline earths and optionally other elements such as aluminum, boron and phosphorous wherein said oxides are present in the following amounts:

| | |
|---|---|
| SiO₂ | 38-57.5 wt %, |
| Na₂O | 16-29 wt %, |
| CaO | 11-25 wt %, |
| Al₂O₃ | 0-3 wt %, |
| B₂O₃ | 0-3 wt %, and |
| P₂O₅ | 0-8 wt %. |

9. The glass ionomer according to claim 8 wherein said bioactive ceramic is selected from the group consisting of S38P8, S45P7, S46P0, S48P2, S51P7, S52PS, S53P4, S55.5P4, S56P6 and S57.5P5.

10. The glass ionomer according to claim 1 wherein said bioactive ceramic is a sol-gel derived silica gel optionally comprising any one or several of elements selected from the group consisting of Al, B, Ca, F, P, K, Mg, N and Ti.

11. The glass ionomer according to claim 10 wherein the bioactive ceramic comprises oxides of silicon, alkalis, alkaline earths and other elements such as phosphorous wherein said components are present in the bioactive ceramic in the following amounts:

| | |
|---|---|
| SiO₂ or Si-gel | 1-100 wt %, |
| Na₂O | 0-45 wt %, |
| K₂O | 0-45 wt % |
| CaO | 0-40 wt %, |
| MgO | 0-40 wt %, and |
| P₂O₅ | 0-60 wt %. |

12. The glass ionomer according to claim 1 wherein said bioactive ceramic is a powder with a particle size of less than 400 μm.

13. The glass ionomer according to claim 12 wherein bioactive ceramic particles in the size range of 1 to 45 μm are included.

14. The glass ionomer according to claim 1 wherein said glass ionomer comprises an additional bioactive agent selected from the group consisting of anti-inflammatory agents, antimicrobial agents, corticosteroids, fluoride, growth factors, heparin, hydroxylapatites, ormosiles, silica gel, tooth whitening agents, vitamins, living cells and any combination thereof.

15. A method for enhancing mineralization of hard tissue of mammals comprising the steps of
a) mixing a bioactive ceramic with an inert biocompatible ceramic to obtain a glass ionomer material,
b) adding a polymer acid and/or polyacid modified resin to said material obtained in step a) to initiate a setting reaction by dissolving and nucleating Ca²⁺ ions and/or by a polymerization reaction,
c) applying said mixture obtained in step b) to where said glass ionomer is to be used for enhancement of mineralization of hard tissue of mammals, and
d) letting said mixture set,
wherein said set glass ionomer enhances mineralization of hard tissue in contact with it.

16. The method according to claim 15 wherein polymer acid added in step b) is selected from the group consisting of polycarboxyl acid, acrylic acid, maleic acid, tartaric acid or their copolymer or any combination thereof.

17. The method according to claim 15 wherein additional components
a) to control physical and/or chemical characteristics, and/or
b) to control appearance of
i) the material obtained in step b) to be applied for use, and/or
ii) the set glass ionomer obtained by the method are added to the material obtained in step a) and/or mixture obtained in step b).

18. The method according to claim 17 wherein said additional components are selected from the group consisting of antimicrobial, bonding, coloring, flow enhancing, reinforcing and suspension enhancing agents, and drugs, growth factors, and preservatives.

19. The method according to claim 17 wherein said additional components are added as granules, fibers, nets or microspheres.

20. The method according to claim 15 wherein the mixture obtained in step b) is applied in step c) for treatment of defects of hard and/or soft tissue.

21. The method according to claim 20 wherein said tissue, with hard and/or soft tissue defects, is selected from the group consisting of maxilla, mandible, tooth, root canal, pulp of a tooth, gingiva, ear, nose, skull, joints, bone and subcutaneous soft tissue.

22. The method according to claim 20 wherein the mixture obtained in step b) is applied into and/or onto said hard and/or soft tissue.

23. The method according to claim 15 wherein said application of step d) is a step of preparation of products selected from the group consisting of implant materials, tissue coating materials, reconstructive parts for tissues, bone augmentation materials and scaffolds for tissue engineering.

24. The method according to claim 23 wherein said product is an injectable material.

25. The method according to claim 24 wherein said product is a solution or a suspension.

26. The method according to claim 25 wherein said product is a material used for coating of teeth and bone.

27. The method according to claim 23 wherein said product is a dental product used
as root canal filling of a tooth or a cavity of a tooth or root of a tooth,
as tooth pulp capping material,
as cementing material of temporary crowns, or
for periodontal defects.

28. The use of the glass ionomer according to any of claims 1 to 14 for the preparation of products intended for treatment of defects of soft and hard tissue, preferably maxilla, mandible, tooth, root canal, pulp of a tooth, gingiva, ear, nose, skull, joints, defects in bone and subcutaneous soft tissue or as a temporary protecting material on soft and hard tissues most preferably for periodontal use.

29. The use of the glass ionomer according to any of claims 1 to 14 for the preparation of products selected from the group consisting of implant materials, tissue coating materials, reconstructive parts for tissues, bone augmentation materials and scaffolds for tissue engineering.

30. The use of the glass ionomer according to any of claims 1 to 14 for the production of an injectable material preferably a solution or a suspension.

31. The use of the glass ionomer according to any of claims 1 to 14 for the production of a material used for coating of teeth and bone.

32. The use of the glass ionomer according to any of claims 1 to 14 for the production of a dental product used
as root canal filling of a tooth or a cavity of a tooth or root of a tooth,
as tooth pulp capping material,
as cementing material of temporary crowns, or
for periodontal defects.
